(19) United States
(12) Patent Application Publication
(10) Pub. No.: US 2011/0144595 A1
(43) Pub. Date: Jun. 16, 2011

(54) INTRAVENOUS DRIP MONITORING METHOD AND RELATED INTRAVENOUS DRIP MONITORING SYSTEM

(76) Inventor: Ting-Yuan Cheng, Taipei (TW)

(21) Appl. No.: 12/791,885

(22) Filed: Jun. 2, 2010

(30) Foreign Application Priority Data
Dec. 11, 2009 (TW) 098142461

(51) Int. Cl. A61M 5/14

(52) U.S. Cl. 604/253

(57) ABSTRACT

The present invention provides an intravenous drip monitoring method and related intravenous drip monitoring system. The intravenous drip monitoring method comprises: providing a background color as a background of a flow regulating device of an intravenous drip; obtaining a plurality of frames in the flow regulating device of the intravenous drip in accordance of a frame rate; performing an image processing operation on the plurality of frames to obtain brightness variations generated by motion drips in the flow regulating device; utilizing the brightness variations generated by the motion drips in the flow regulating device to detect a dropping frequency of the intravenous drip in a first detecting area of the flow regulating device; and utilizing the brightness variations generated by the motion drips in the flow regulating device to detect a liquid horizontal height of the flow regulating device in a second detecting area of the flow regulating device.

### BACKGROUND OF THE INVENTION

1. Field of the Invention

The present invention relates to an intravenous drip monitoring method and a related intravenous drip monitoring system, and more particularly, to an intravenous drip monitoring method and a related intravenous drip monitoring system capable of precisely monitoring dripping conditions of an intravenous drip in order to send corresponding warning messages in accordance with different dripping conditions, and the intravenous drip monitoring method and the related intravenous drip monitoring system disclosed by the present invention have advantages of high efficiency and low cost.

2. Description of the Prior Art

In general, the conventional intravenous drip monitoring methods and related intravenous drip monitoring systems use sensors and measuring circuit to realize the intravenous drip monitoring function. However, the conventional intravenous drip monitoring methods and related intravenous drip monitoring systems cost higher and the effect thereof is not very good. For example, the conventional intravenous drip monitoring methods and related intravenous drip monitoring systems can not precisely monitor a dripping condition of an intravenous drip in order to send corresponding warning messages in accordance with different dripping conditions.

### SUMMARY OF THE INVENTION

It is therefore one of the objectives of the present invention to provide an intravenous drip monitoring method and a related intravenous drip monitoring system capable of precisely monitoring dripping conditions of an intravenous drip in order to send corresponding warning messages in accordance with different dripping conditions, and the intravenous drip monitoring method and the related intravenous drip monitoring system disclosed by the present invention have advantages of high efficiency and low cost, so as to solve the above problem.

In accordance with an embodiment of the present invention, an intravenous drip monitoring method is disclosed. The intravenous drip monitoring method comprises: providing a background color as a background of a flow regulating device of an intravenous drip; obtaining a plurality of frames in the flow regulating device of the intravenous drip in accordance of a frame rate; performing an image processing operation on the plurality of frames to obtain brightness variations generated by motion drips in the flow regulating device; utilizing the brightness variations generated by the motion drips in the flow regulating device to detect a dropping frequency of the intravenous drip in a first detecting area of the flow regulating device; and utilizing the brightness variations generated by the motion drips in the flow regulating device to detect a liquid horizontal height of the flow regulating device in a second detecting area of the flow regulating device.

In accordance with an embodiment of the present invention, an intravenous drip monitoring system is further disclosed. The intravenous drip monitoring system comprises: an image obtaining device, a color plate, and an image processing device. The image obtaining device is positioned in a side of a flow regulating device of an intravenous drip, and utilized for obtaining a plurality of frames in the flow regulating device of the intravenous drip. The color plate is positioned in a side of a flow regulating device of an intravenous drip, and utilized for providing a background color as a background of a flow regulating device of an intravenous drip. The image processing device is coupled to the image obtaining device, and utilized for: obtaining a plurality of frames in the flow regulating device of the intravenous drip in accordance of a frame rate; performing an image processing operation on the plurality of frames to obtain brightness variations generated by motion drips in the flow regulating device; utilizing the brightness variations generated by the motion drips in the flow regulating device to detect a dropping frequency of the intravenous drip in a first detecting area of the flow regulating device; and utilizing the brightness variations generated by the motion drips in the flow regulating device to detect a liquid horizontal height of the flow regulating device in a second detecting area of the flow regulating device.

Briefly summarized, the intravenous drip monitoring method and the related intravenous drip monitoring system disclosed by the present invention are capable of precisely monitoring dripping conditions of an intravenous drip in order to send corresponding warning messages in accordance with different dripping conditions, and the intravenous drip monitoring method and the related intravenous drip monitoring system disclosed by the present invention have advantages of high efficiency and low cost.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows a simplified block diagram of an intravenous drip monitoring system in accordance with an embodiment of the present invention.

FIG. 2 is a flowchart illustrating an intravenous drip monitoring method employed for the intravenous drip monitoring system shown in FIG. 1 in accordance with an exemplary embodiment of the present invention.

FIG. 3 shows a simplified diagram of providing a background color as a background of a flow regulating device of an intravenous drip at first in the Step 210 as shown in FIG. 2.

FIG. 4 shows a simplified diagram of obtaining a plurality of frames F1, F2, . . . , Fn in the flow regulating device of the intravenous drip in accordance of a frame rate in the Step 220 as shown in FIG. 2.

FIG. 5 shows a simplified diagram of performing an image processing operation on the plurality of frames F1, F2, . . . , Fn to obtain brightness variations generated by motion drips in the flow regulating device in the Step 230 as shown in FIG. 2.

FIG. 6 shows a simplified diagram of utilizing the brightness variations generated by the motion drips in the flow regulating device to define a first detecting area and a second detecting area in the Step 240 as shown in FIG. 2.

FIG. 7 is a flowchart illustrating an exemplary embodiment of the Step 240 as shown in FIG. 2.

FIG. 8 is a flowchart illustrating a first exemplary embodiment of the Step 250 as shown in FIG. 2.

FIG. 9 shows a simplified diagram of defining a detecting sub-area in the first detecting area in the Step 251 as shown in FIG. 8.

FIG. 10 shows a simplified timing diagram of labeling the plurality of time points as a plurality of peak occurring time points when observing the numbers of motion blocks in the detecting sub-area respectively exceeding a threshold TH at a plurality of time points in the Step 255 as shown in FIG. 8.

FIG. 11 is a flowchart illustrating a second exemplary embodiment of the Step 250 as shown in FIG. 2.

FIG. 12 shows a simplified diagram of defining three detecting sub-areas in the first detecting area in the Step 251 as shown in FIG. 11.

FIG. 13 shows a simplified timing diagram of labeling the plurality of time points as a plurality of first peak occurring time points when observing the numbers of motion blocks in the detecting sub-area respectively exceeding a threshold TH at a plurality of time points in the Step 253 as shown in FIG. 11.

FIG. 14 is a flowchart illustrating an exemplary embodiment of the Step 257 as shown in FIG. 11.

FIG. 15 shows a simplified diagram of setting a first observing window Dwin1 at each time point spaced at an interval of the average distance D in the Step 257a as shown in FIG. 14.

FIG. 16 is a finite state machine diagram illustrating an exemplary embodiment of the Step 257b as shown in FIG. 14.

FIG. 17 shows a simplified diagram of using a phase lock loop (PLL) to calculate an actual distance Da per two peak occurring time points to calculating an actual average distance Day per two peak occurring time points, setting a second observing window Dwin2 at each time point spaced at an interval of the actual average distance Day, and determining whether a peak occurring time point P appears in a current second observing window Dwin2 in the state S2 as shown in FIG. 16.

FIG. 18 is a flowchart illustrating an exemplary embodiment of the Step 260 as shown in FIG. 2.

FIG. 19 shows a simplified diagram of defining three detecting sub-areas in the second detecting area from top to bottom in the Step 261 as shown in FIG. 18.

### DETAILED DESCRIPTION

Certain terms are used throughout the following description and the claims to refer to particular system components. As one skilled in the art will appreciate, manufacturers may refer to a component by different names. This document does not intend to distinguish between components that differ in name but not function. In the following discussion and in the claims, the terms “include”, “including”, “comprise”, and “comprising” are used in an open-ended fashion, and thus should be interpreted to mean “including, but not limited to . . . ”. The terms “couple” and “coupled” are intended to mean either an indirect or a direct electrical connection. Thus, if a first device couples to a second device, that connection may be through a direct electrical connection, or through an indirect electrical connection via other devices and connections.

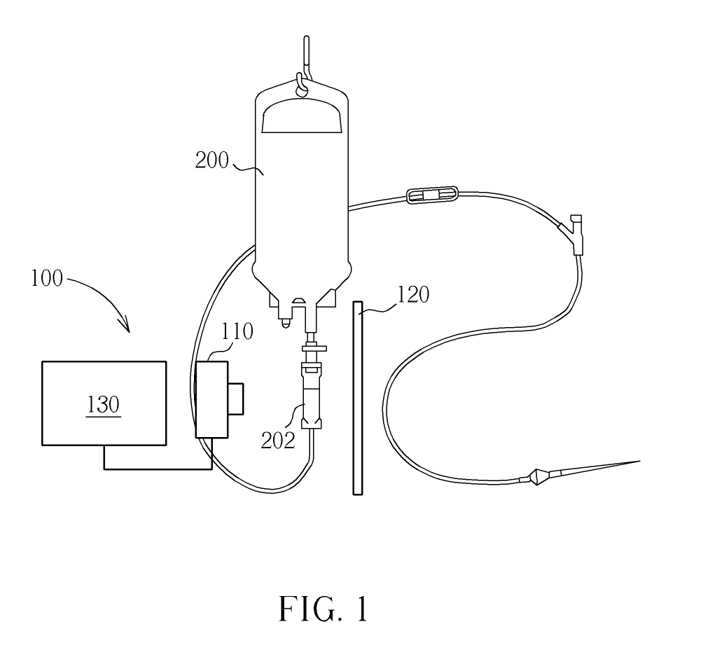

Please refer to FIG. 1. FIG. 1 shows a simplified block diagram of an intravenous drip monitoring system 100 in accordance with an embodiment of the present invention. As shown in

FIG. 1, the intravenous drip monitoring system 100 comprises: an image obtaining device 110, a color plate 120, and an image processing device 130. The image obtaining device 110 is positioned in a side of a flow regulating device 202 of an intravenous drip 200, and utilized for obtaining a plurality of frames in the flow regulating device 202 of the intravenous drip 200. The color plate 120 is positioned in a side of a flow regulating device 202 of an intravenous drip 200, and utilized for providing a background color (such as a red color that does not reflect light) as a background of a flow regulating device 202 of an intravenous drip 200. The image processing device 130 is coupled to the image obtaining device 110, and utilized for: obtaining a plurality of frames in the flow regulating device 202 of the intravenous drip 200 in accordance of a frame rate; performing an image processing operation on the plurality of frames to obtain brightness variations generated by motion drips in the flow regulating device 202; utilizing the brightness variations generated by the motion drips in the flow regulating device 202 to detect a dropping frequency of the intravenous drip 200 in a first detecting area of the flow regulating device 202; and utilizing the brightness variations generated by the motion drips in the flow regulating device 202 to detect a liquid horizontal height of the flow regulating device 202 in a second detecting area of the flow regulating device 202. In addition, the image processing device 130 can be further utilized for utilizing the brightness variations generated by the motion drips in the flow regulating device 202 to define the first detecting area and the second detecting area, and utilized for determining whether to output a warning message in accordance with the dropping frequency and the liquid horizontal height, wherein the image processing device 110 can be a digital camera or a web cam.

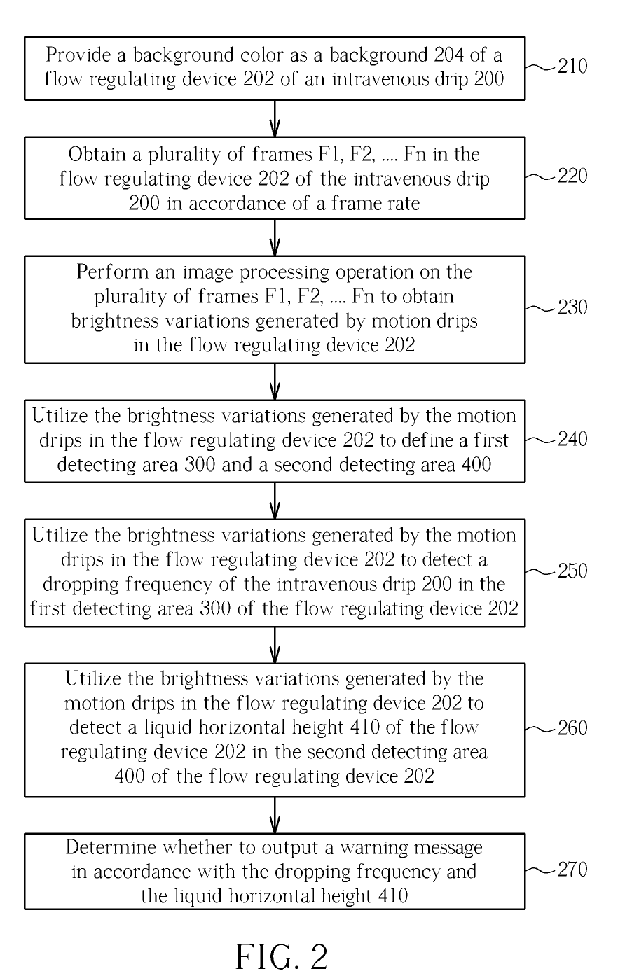

Please refer to FIG. 2. FIG. 2 is a flowchart illustrating an intravenous drip monitoring method employed for the intravenous drip monitoring system 100 shown in FIG. 1 in accordance with an exemplary embodiment of the present invention. Please note that, provided substantially the same result is achieved, the steps of the flow shown in FIG. 2 need not be in the exact order shown and need not be contiguous; that is, other steps can be intermediate. The intravenous drip monitoring method comprises the following steps:

Step 210: Provide a background color as a background 204 of a flow regulating device 202 of an intravenous drip 200.

Step 220: Obtain a plurality of frames F1, F2, . . . , Fn in the flow regulating device 202 of the intravenous drip 200 in accordance of a frame rate.

Step 230: Perform an image processing operation on the plurality of frames F1, F2, . . . , Fn to obtain brightness variations generated by motion drips in the flow regulating device 202.

Step 240: Utilize the brightness variations generated by the motion drips in the flow regulating device 202 to define a first detecting area 300 and a second detecting area 400.

Step 250: Utilize the brightness variations generated by the motion drips in the flow regulating device 202 to detect a dropping frequency of the intravenous drip 200 in the first detecting area 300 of the flow regulating device 202.

Step 260: Utilize the brightness variations generated by the motion drips in the flow regulating device 202 to detect a liquid horizontal height 410 of the flow regulating device 202 in the second detecting area 400 of the flow regulating device 202.

Step 270: Determine whether to output a warning message in accordance with the dropping frequency and the liquid horizontal height 410.

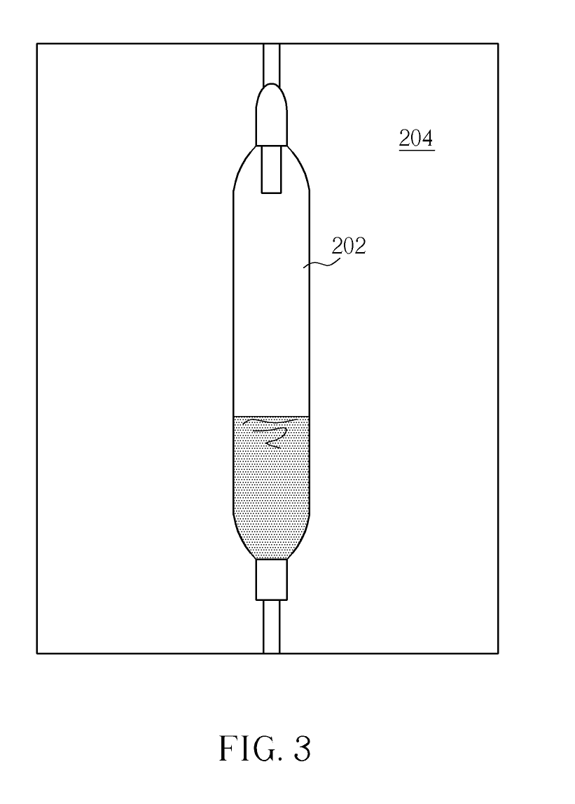

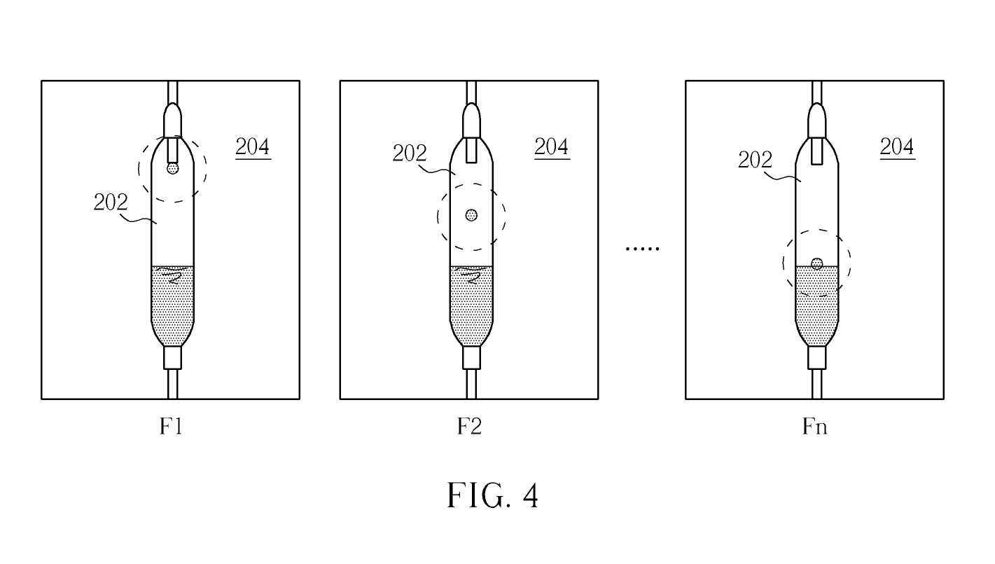

As shown in FIG. 2, in the Step 210, the intravenous drip monitoring method provides a background color as a background 240 of a flow regulating device 202 of an intravenous drip 200 at first, as shown in FIG. 3. Next, please refer to FIG. 2 again. In the Step 220, the intravenous drip monitoring method obtains a plurality of frames F1, F2, . . . , Fn in the flow regulating device 202 of the intravenous drip 200 in accordance of a frame rate, as shown in FIG. 4.

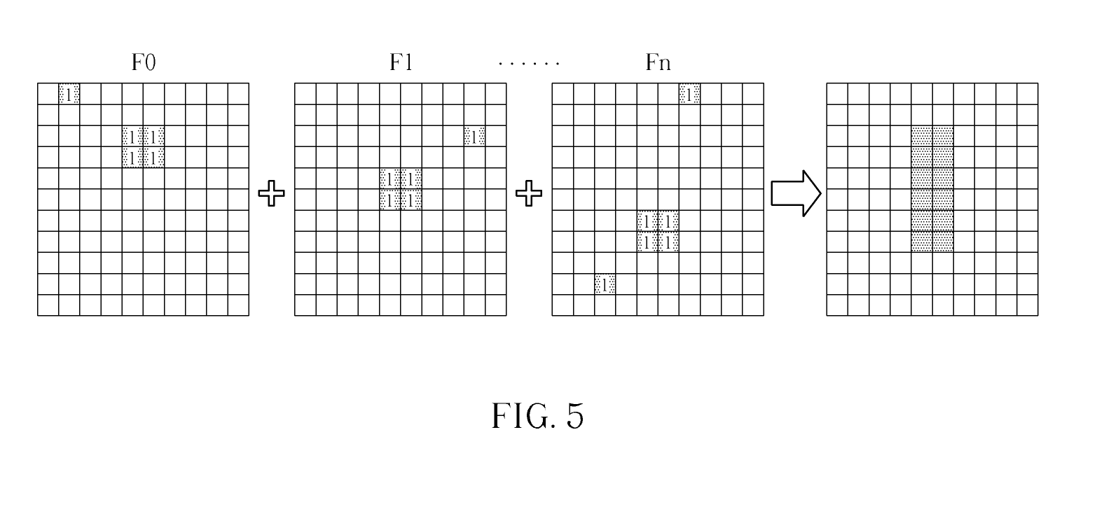

Next, please refer to FIG. 2 again. In the Step 230, the intravenous drip monitoring method performs an image processing operation on the plurality of frames F1, F2, . . . , Fn to obtain brightness variations generated by motion drips in the flow regulating device 202, as shown in FIG. 5. The bright spots are labeled as 1, and the positions of most bright spots are the positions that the motion drips pass by. Thus, summing the plurality of frames F1, F2, . . . , Fn can obtain a complete area that the motion drips in the flow regulating device 202 pass by.

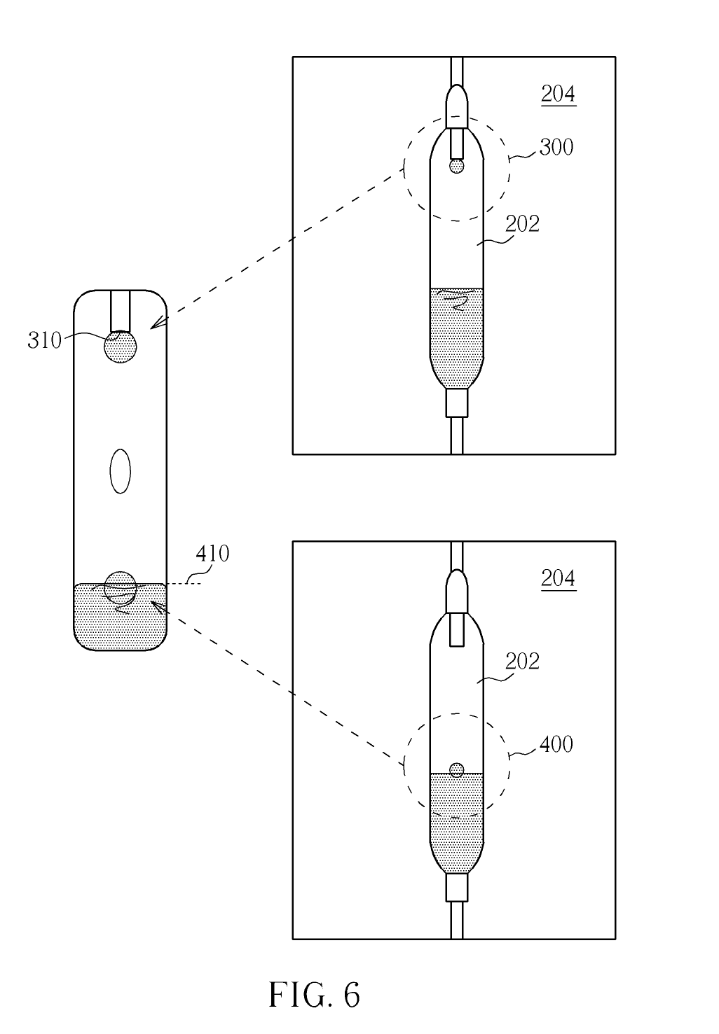

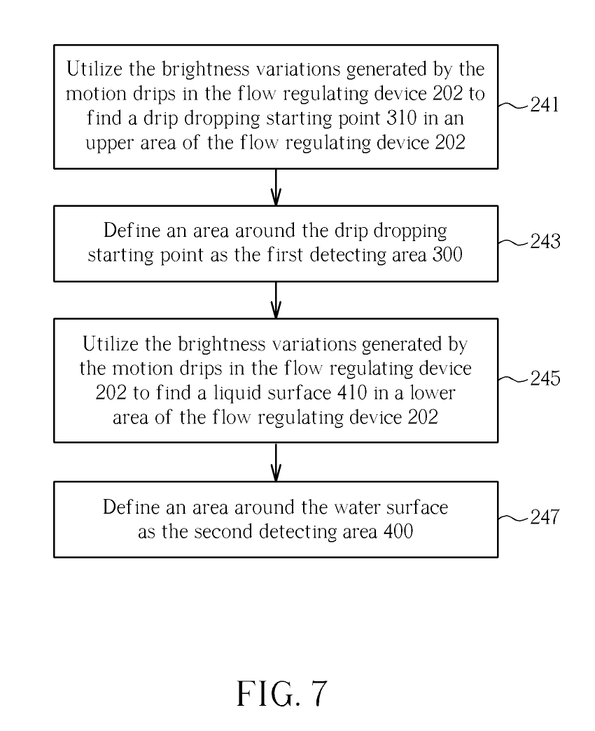

Next, please refer to FIG. 2 again. In the Step 240, the intravenous drip monitoring method utilizes the brightness variations generated by the motion drips in the flow regulating device 202 to define a first detecting area 300 and a second detecting area 400, as shown in FIG. 6. In addition, please refer to FIG. 7. FIG. 7 is a flowchart illustrating an exemplary embodiment of the Step 240 as shown in FIG. 2. As shown in FIG. 7, the step of utilizing the brightness variations generated by the motion drips in the flow regulating device 202 to define the first detecting area 300 and the second detecting area 400 can comprise: Step 241: utilizing the brightness variations generated by the motion drips in the flow regulating device 202 to find a drip dropping starting point 310 in an upper area of the flow regulating device 202; Step 243: defining an area around the drip dropping starting point as the first detecting area 300; Step 245:

utilizing the brightness variations generated by the motion drips in the flow regulating device 202 to find a liquid surface 410 in a lower area of the flow regulating device 202; and Step 247: defining an area around the water surface as the second detecting area 400.

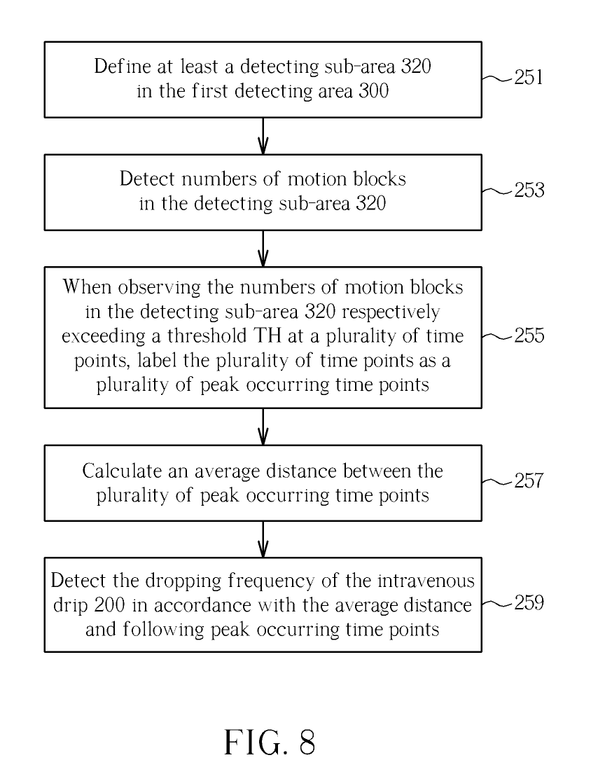

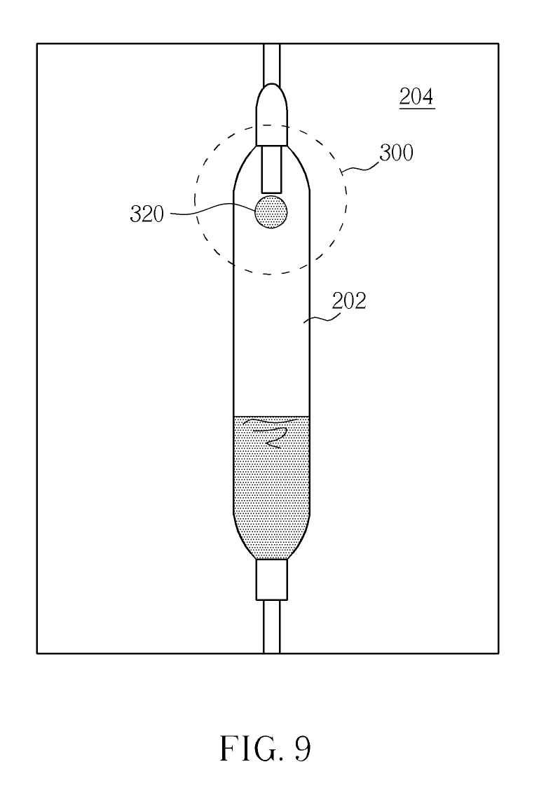

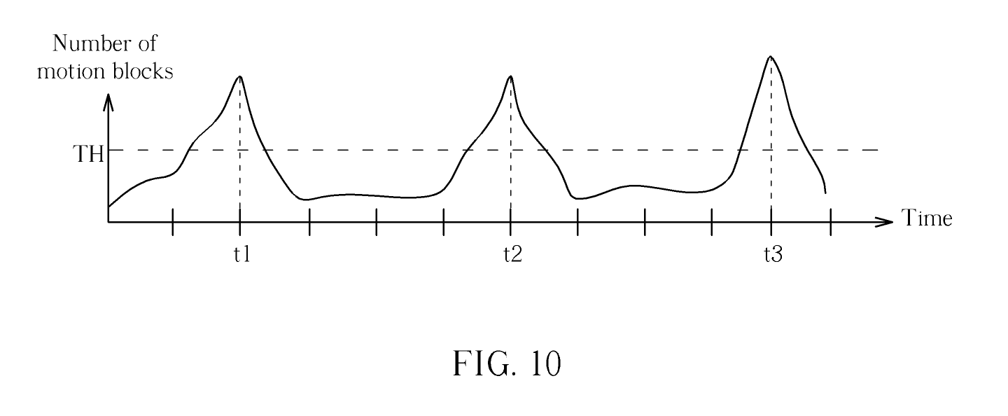

Next, please refer to FIG. 2 again. In the Step 250, the intravenous drip monitoring method utilizes the brightness variations generated by the motion drips in the flow regulating device 202 to detect a dropping frequency of the intravenous drip 200 in the first detecting area 300 of the flow regulating device 202. In addition, please refer to FIG. 8. FIG. 8 is a flowchart illustrating a first exemplary embodiment of the Step 250 as shown in FIG. 2. As shown in FIG. 8, the step of utilizing the brightness variations generated by the motion drips in the flow regulating device 202 to detect a dropping frequency of the intravenous drip 200 in the first detecting area 300 of the flow regulating device 202 can comprise: Step 251: defining a detecting sub-area 320 in the first detecting area 300 (as shown in FIG. 9); Step 253: detecting numbers of motion blocks in the detecting sub-area 320; Step 255: when observing the numbers of motion blocks in the detecting sub-area 320 respectively exceeding a threshold TH at a plurality of time points (such as t1, t2, and t3 in FIG. 10), labeling the plurality of time points as a plurality of peak occurring time points (as shown in FIG. 10); Step 257: calculating an average distance between the plurality of peak occurring time points; and Step 259: detecting the dropping frequency of the intravenous drip 200 in accordance with the average distance and following peak occurring time points. The threshold TH is set by observing the numbers generated by the motion drips in the flow regulating device passing the detecting sub-area 320 during a specific time period.

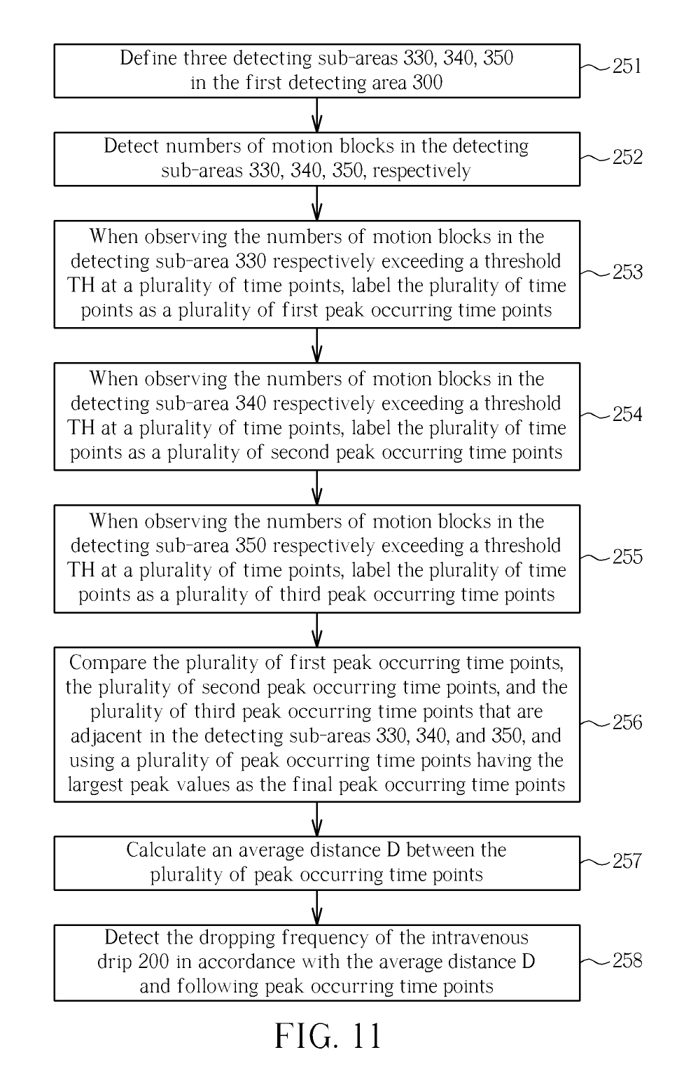

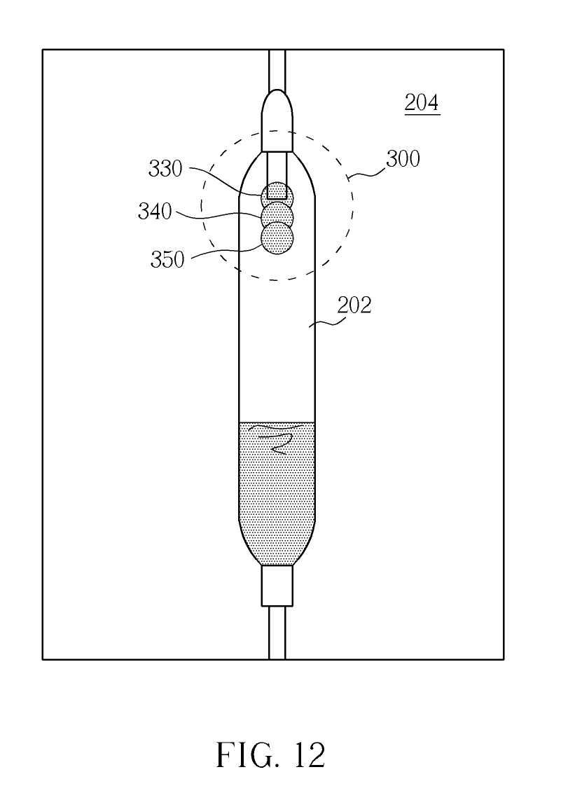

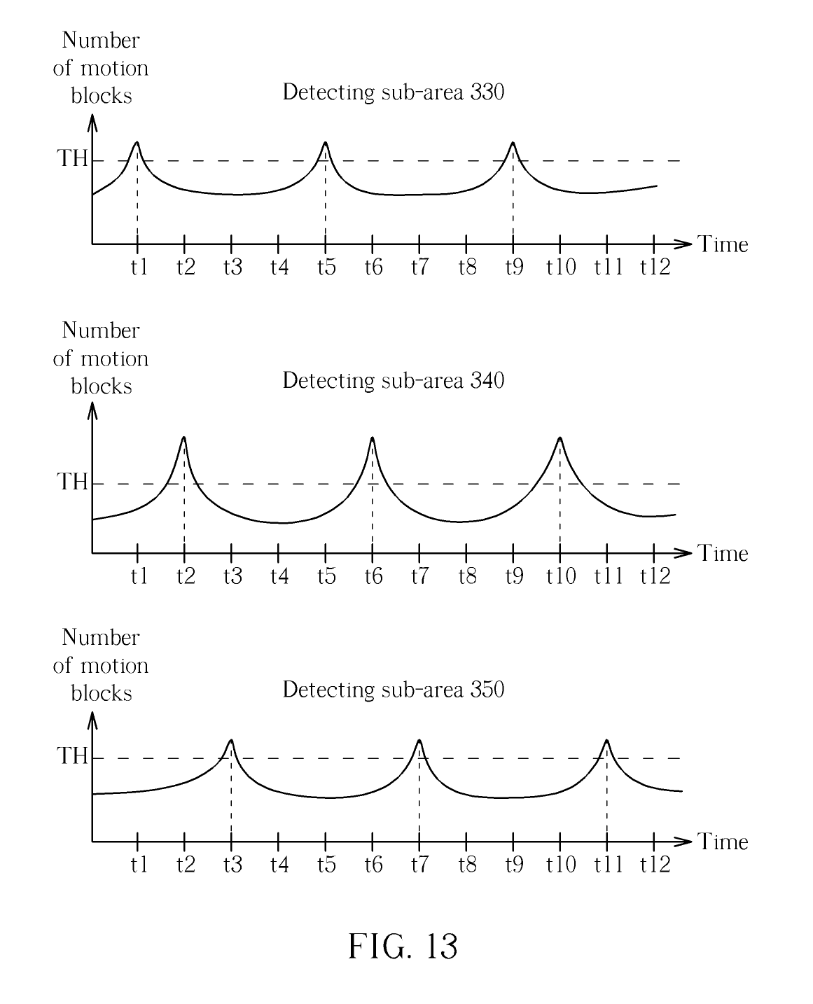

In addition, please refer to FIG. 11. FIG. 11 is a flowchart illustrating a second exemplary embodiment of the Step 250 as shown in FIG. 2. As shown in FIG. 11, the step of utilizing the brightness variations generated by the motion drips in the flow regulating device 202 to detect a dropping frequency of the intravenous drip 200 in the first detecting area 300 of the flow regulating device 202 can comprise: Step 251: defining three detecting sub-areas 330, 340, 350 in the first detecting area 300 (as shown in FIG. 12); Step 252: detecting numbers of motion blocks in the detecting sub-areas 330, 340, 350, respectively; Step 253: when observing the numbers of motion blocks in the detecting sub-area 330 respectively exceeding a threshold TH at a plurality of time points (such as t1, t5, and t9 in FIG. 13), labeling the plurality of time points as a plurality of first peak occurring time points (as shown in FIG. 13); Step 254: when observing the numbers of motion blocks in the detecting sub-area 340 respectively exceeding a threshold TH at a plurality of time points (such as t2, t6, and t10 in FIG. 13), labeling the plurality of time points as a plurality of second peak occurring time points (as shown in FIG. 13); Step 255: when observing the numbers of motion blocks in the detecting sub-area 350 respectively exceeding a threshold TH at a plurality of time points (such as t3, t7, and t11 in FIG. 13), labeling the plurality of time points as a plurality of third peak occurring time points (as shown in FIG. 13); Step 256: comparing the plurality of first peak occurring time points, the plurality of second peak occurring time points, and the plurality of third peak occurring time points that are adjacent in the detecting sub-areas 330, 340, and 350, and using a plurality of peak occurring time points having the largest peak values (such as t2, t6, and t10 in FIG. 13) as the final peak occurring time points; Step 257: calculating an average distance D between the plurality of peak occurring time points; and Step 258: detecting the dropping frequency of the intravenous drip 200 in accordance with the average distance D and following peak occurring time points. The threshold TH is set by observing the numbers generated by the motion drips in the flow regulating device passing the detecting sub-areas 330, 340, and 350 during a specific time period. Herein, please note that the above embodiment is only for an illustrative purpose and is not meant to be a limitation of the present invention. For example, the number of the detecting sub-areas defined in the first detecting area 300 can be changed in accordance with different designs.

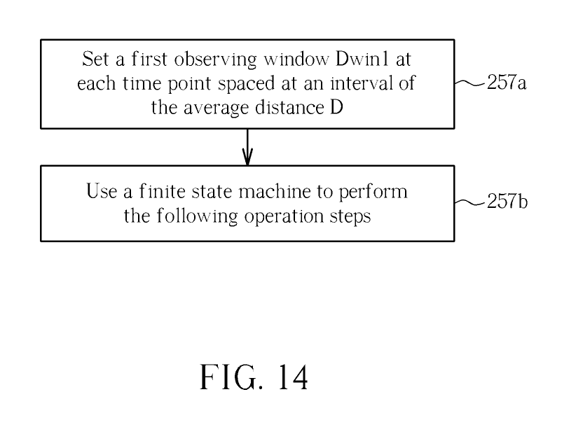

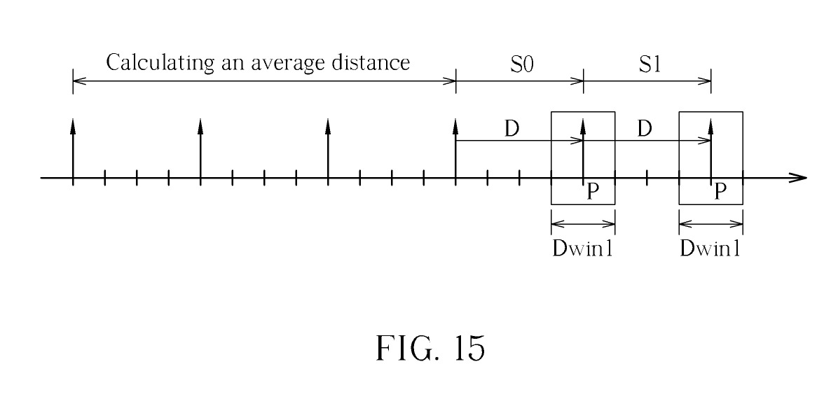

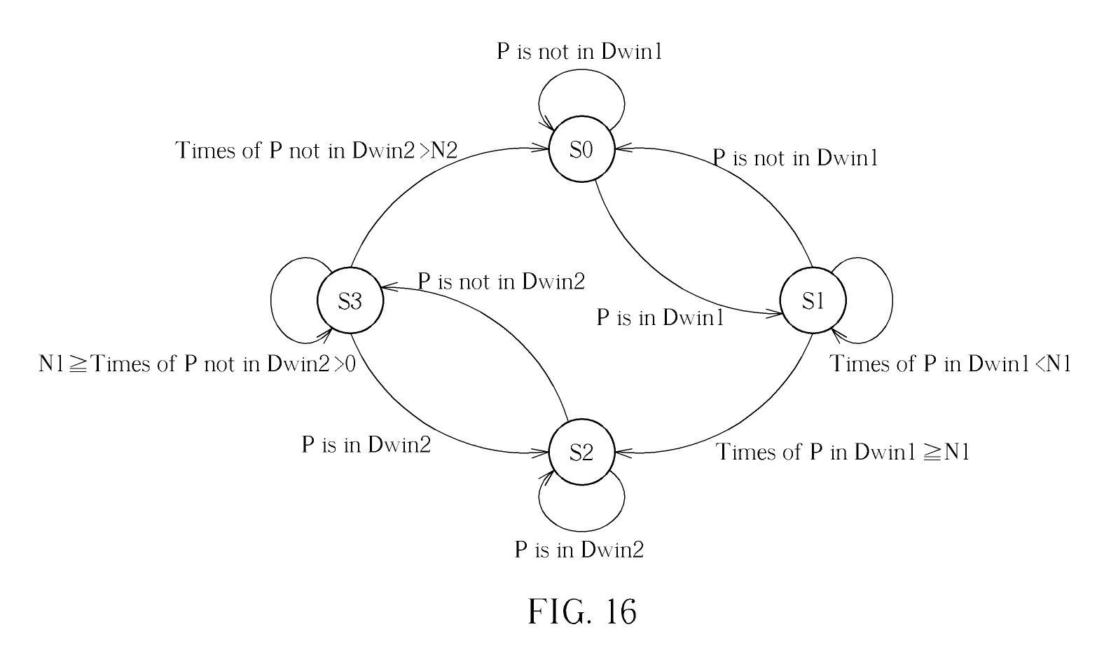

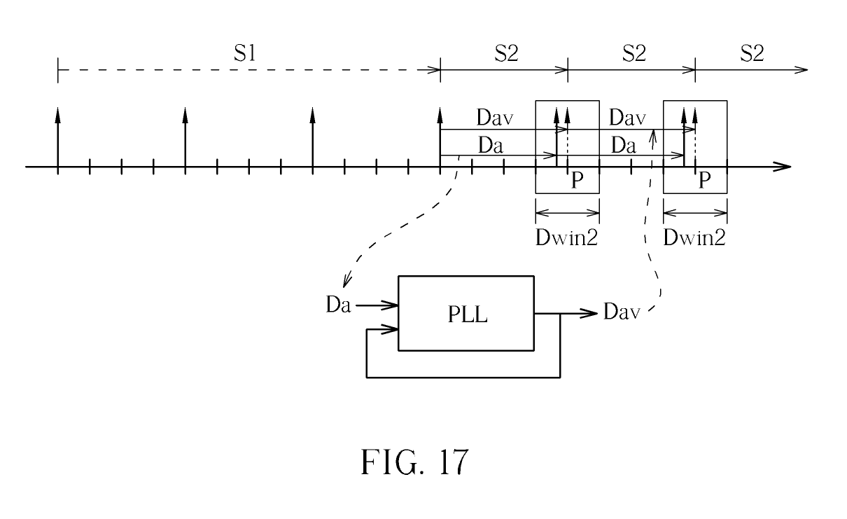

In addition, please refer to FIG. 14. FIG. 14 is a flowchart illustrating an exemplary embodiment of the Step 257 as shown in FIG. 11. As shown in FIG. 11, the step of detecting the dropping frequency of the intravenous drip 200 in accordance with the average distance D and the following peak occurring time points can comprise: Step 257a: setting a first observing window Dwin1 at each time point spaced at an interval of the average distance D (as shown in FIG. 15); and Step 257b: using a finite state machine to perform the following operation steps. Please refer to FIG. 16. FIG. 16 is a finite state machine diagram illustrating an exemplary embodiment of the Step 257b as shown in FIG. 14. The illustration of using the finite state machine to perform the following operation steps are as follows:
in a first state S0, determining whether a peak occurring time point P appears in a current first observing window Dwin1, wherein:
when the peak occurring time point P does not appear in the current first observing window Dwin1, keeping in the first state SO and adjusting the frame rate, and calculating the average distance again; and
when the peak occurring time point P appears in the current first observing window Dwin1, entering a second state S1;
in the second state S1, determining whether a peak occurring time point P appears in a current first observing window Dwin1 and determining whether a first number of accumulating times of the peak occurring time points greater than a first predetermined number N1, wherein:
when the peak occurring time point P does not appear in the current first observing window Dwin1, go back to the first state SO and calculating the average distance again;
when the peak occurring time point P appears in the current first observing window Dwin1, and the first number of accumulating times of the peak occurring time points is smaller than the first predetermined number N1, keeping in the second state S1; and
when the peak occurring time point P appears in the current first observing window Dwin1, and the first number of accumulating times of the peak occurring time points is not smaller than the first predetermined number N1, entering a third state S2;
in the third state S2, using a phase lock loop (PLL) to calculate an actual distance Da per two peak occurring time points to calculating an actual average distance Day per two peak occurring time points, setting a second observing window Dwin2 at each time point spaced at an interval of the actual average distance Day, and determining whether a peak occurring time point P appears in a current second observing window Dwin2 (as shown in FIG. 17), wherein:
when the peak occurring time point P does not appear in the current second observing window Dwin2, entering a fourth state S3; and
when the peak occurring time point P appears in the current second observing window Dwin2, keeping in the third state S2;
in the fourth state S3, determining whether a peak occurring time point P appears in a current second observing window Dwin2 and determining whether a second number of accumulating times of the peak occurring time points greater than a second predetermined number N2, wherein:
when the peak occurring time point P does not appear in the current second observing window Dwin2, go back to the third state S2;
when the peak occurring time point P does not appear in the current second observing window Dwin2, and the second number of accumulating times of the peak occurring time points is greater than the second predetermined number N2, entering the first state S0; and
when the peak occurring time point P does not appear in the current second observing window Dwin2, and the second number of accumulating times of the peak occurring time points is not greater than the second predetermined number N2, keeping in the fourth state S3.

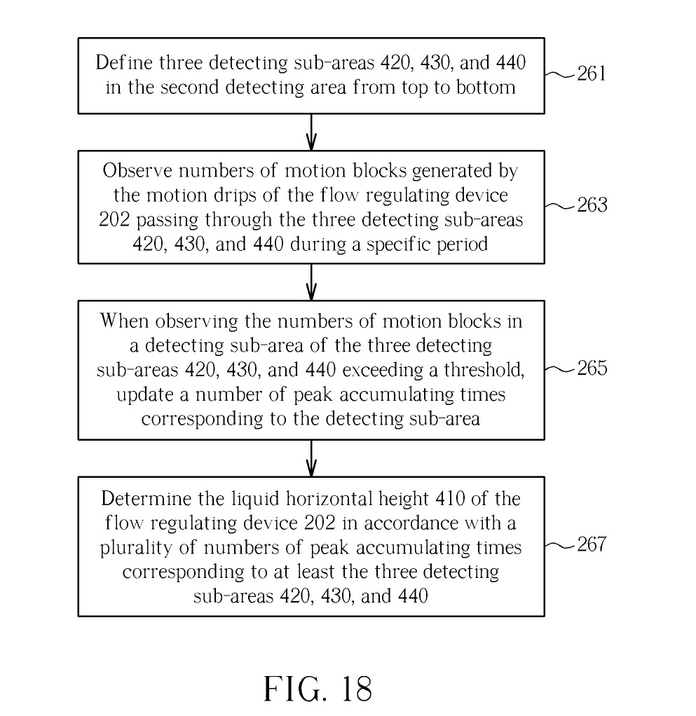

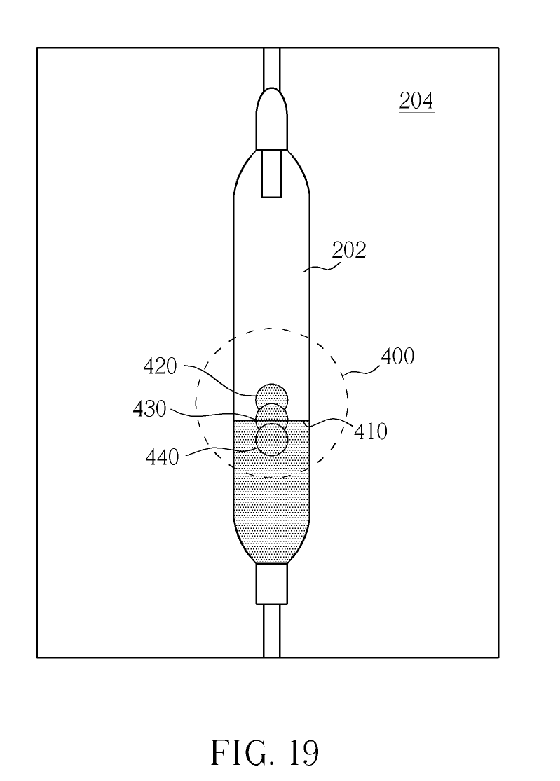

Next, please refer to FIG. 2 again. In the Step 260, the intravenous drip monitoring method utilizes the brightness variations generated by the motion drips in the flow regulating device 202 to detect a liquid horizontal height 410 of the flow regulating device 202 in the second detecting area 400 of the flow regulating device 202. In addition, please refer to FIG. 18. FIG. 18 is a flowchart illustrating an exemplary embodiment of the Step 260 as shown in FIG. 2. As shown in FIG. 18, the step of utilizing the brightness variations generated by the motion drips in the flow regulating device 202 to detect the liquid horizontal height 410 of the flow regulating device 202 in the second detecting area 400 of the flow regulating device 202 can comprise: Step 261: defining three detecting sub-areas 420, 430, and 440 (as shown in FIG. 19) in the second detecting area from top to bottom; Step 263: observing numbers of motion blocks generated by the motion drips of the flow regulating device 202 passing through the three detecting sub-areas 420, 430, and 440 during a specific period; Step 265: when observing the numbers of motion blocks in a detecting sub-area of the three detecting sub-areas 420, 430, and 440 exceeding a threshold, updating a number of peak accumulating times corresponding to the detecting sub-area; and Step 267: determining the liquid horizontal height 410 of the flow regulating device 202 in accordance with a plurality of numbers of peak accumulating times corresponding to at least the three detecting sub-areas 420, 430, and 440. In addition, in an exemplary embodiment of the present invention, the step of determining the liquid horizontal height 410 of the flow regulating device 202 can comprise: performing a weighted averages process to determine the liquid horizontal height 410 of the flow regulating device 202 in accordance with the plurality of numbers of peak accumulating times and heights of the three detecting sub-areas 420, 430, and 440. In another exemplary embodiment of the present invention, the step of determining the liquid horizontal height 410 of the flow regulating device 202 can comprise: determining the liquid horizontal height 410 of the flow regulating device 202 in accordance with a detecting sub-area having a largest peak accumulating time of the three detecting sub-areas 420, 430, and 440. Herein, please note that the above embodiment is only for an illustrative purpose and is not meant to be a limitation of the present invention. For example, the number of the detecting sub-areas defined in the second detecting area 400 can be changed in accordance with different designs.

Next, please refer to FIG. 2 again. In the Step 270, the intravenous drip monitoring method determines whether to output a warning message in accordance with the dropping frequency and the liquid horizontal height 410. When remaining in the third state S2 and an actual average distance Day between every two peak occurring time points is smaller than a determined distance, outputting a warning message to warn that the dropping frequency is too high. When remaining in the third state S2 and an actual average distance Day between every two peak occurring time points is greater than a determined distance, outputting a warning message to warn that the dropping frequency is too low. When entering to the fourth state S3 from the first state S0, outputting a warning message to warn that the intravenous drip 200 stops to drip. When entering to the fourth state S3 from the first state S0 and the liquid horizontal height 410 of the flow regulating device 202 is lower than a determined height, outputting a warning message to warn that the intravenous drip 200 is empty. When entering to the fourth state S3 from the first state S0 and the liquid horizontal height 410 of the flow regulating device 202 does not have any variation, outputting a warning message to warn that a tube of the intravenous drip 200 is jammed.

Briefly summarized, the intravenous drip monitoring method and the related intravenous drip monitoring system disclosed by the present invention are capable of precisely monitoring dripping conditions of an intravenous drip in order to send corresponding warning messages in accordance with different dripping conditions, and the intravenous drip monitoring method and the related intravenous drip monitoring system disclosed by the present invention have advantages of high efficiency and low cost.

Those skilled in the art will readily observe that numerous modifications and alterations of the device and method may be made while retaining the teachings of the invention.

What is claimed is:

1. An intravenous drip monitoring method, comprising:
providing a background color as a background of a flow regulating device of an intravenous drip;
obtaining a plurality of frames in the flow regulating device of the intravenous drip in accordance of a frame rate;
performing an image processing operation on the plurality of frames to obtain brightness variations generated by motion drips in the flow regulating device;
utilizing the brightness variations generated by the motion drips in the flow regulating device to detect a dropping frequency of the intravenous drip in a first detecting area of the flow regulating device; and
utilizing the brightness variations generated by the motion drips in the flow regulating device to detect a liquid horizontal height of the flow regulating device in a second detecting area of the flow regulating device.

2. The intravenous drip monitoring method of claim 1, further comprising:
utilizing the brightness variations generated by the motion drips in the flow regulating device to define the first detecting area and the second detecting area.

3. The intravenous drip monitoring method of claim 2, wherein the step of utilizing the brightness variations generated by the motion drips in the flow regulating device to define the first detecting area and the second detecting area comprises:
utilizing the brightness variations generated by the motion drips in the flow regulating device to find a drip dropping starting point in an upper area of the flow regulating device;
defining an area around the drip dropping starting point as the first detecting area;
utilizing the brightness variations generated by the motion drips in the flow regulating device to find a liquid surface in a lower area of the flow regulating device; and
defining an area around the water surface as the second detecting area.

4. The intravenous drip monitoring method of claim 1, wherein the step of utilizing the brightness variations generated by the motion drips in the flow regulating device to detect the dropping frequency of the intravenous drip in a first detecting area of the flow regulating device comprises:
defining at least a detecting sub-area in the first detecting area;
detecting numbers of motion blocks in the detecting sub-area;
when observing the numbers of motion blocks respectively exceeding a threshold at a plurality of time points, labeling the plurality of time points as a plurality of peak occurring time points;
calculating an average distance between the plurality of peak occurring time points; and
detecting the dropping frequency of the intravenous drip in accordance with the average distance and following peak occurring time points.

5. The intravenous drip monitoring method of claim 4, wherein the step of detecting the dropping frequency of the intravenous drip in accordance with the average distance and the following peak occurring time points comprises:
setting a first observing window at each time point spaced at an interval of the average distance;
in a first state, determining whether a peak occurring time point appears in a current first observing window, wherein:
when the peak occurring time point does not appear in the current first observing window, keeping in the first state and adjusting the frame rate, and calculating the average distance again; and
when the peak occurring time point appears in the current first observing window, entering a second state;
in the second state, determining whether a peak occurring time point appears in a current first observing window and determining whether a first number of accumulating times of the peak occurring time points greater than a first predetermined number, wherein:
when the peak occurring time point does not appear in the current first observing window, go back to the first state and calculating the average distance again;
when the peak occurring time point appears in the current first observing window, and the first number of accumulating times of the peak occurring time points is smaller than the first predetermined number, keeping in the second state; and
when the peak occurring time point appears in the current first observing window, and the first number of accumulating times of the peak occurring time points is not smaller than the first predetermined number, entering a third state;
in the third state, calculating an actual distance per two peak occurring time points to calculating an actual average distance per two peak occurring time points, setting a second observing window at each time point spaced at an interval of the actual average distance, and determining whether a peak occurring time point appears in a current second observing window, wherein:
when the peak occurring time point does not appear in the current second observing window, entering a fourth state; and
when the peak occurring time point appears in the current second observing window, keeping in the third state;
in the fourth state, determining whether a peak occurring time point appears in a current second observing window and determining whether a second number of accumulating times of the peak occurring time points greater than a second predetermined number, wherein:
when the peak occurring time point does not appear in the current second observing window, go back to the third state;
when the peak occurring time point does not appear in the current second observing window, and the second number of accumulating times of the peak occurring time points is greater than the second predetermined number, entering the first state; and
when the peak occurring time point does not appear in the current second observing window, and the second number of accumulating times of the peak occurring time points is not greater than the second predetermined number, keeping in the fourth state.

6. The intravenous drip monitoring method of claim 5, wherein the step of utilizing the brightness variations generated by the motion drips in the flow regulating device to detect the liquid horizontal height of the flow regulating device in a second detecting area of the flow regulating device comprises:
defining a plurality of detecting sub-areas in the second detecting area from top to bottom;
observing numbers of motion blocks generated by the motion drips of the flow regulating device passing through the plurality of detecting sub-areas during a specific period;
when observing the numbers of motion blocks in a detecting sub-area of the plurality of detecting sub-areas exceeding a threshold, updating a number of peak accumulating times corresponding to the detecting sub-area; and
determining the liquid horizontal height of the flow regulating device in accordance with a plurality of numbers of peak accumulating times corresponding to at least the plurality of detecting sub-areas.

7. The intravenous drip monitoring method of claim 6, wherein the step of determining the liquid horizontal height of the flow regulating device comprises:
performing a weighted averages process to determine the liquid horizontal height of the flow regulating device in accordance with the plurality of numbers of peak accumulating times and heights of the plurality of detecting sub-areas.

8. The intravenous drip monitoring method of claim 6, wherein the step of determining the liquid horizontal height of the flow regulating device comprises:
determining the liquid horizontal height of the flow regulating device in accordance with a detecting sub-area having a largest peak accumulating time of the plurality of detecting sub-areas.

9. The intravenous drip monitoring method of claim 6, further comprising:
determining whether to output a warning message in accordance with the dropping frequency and the liquid horizontal height.

10. The intravenous drip monitoring method of claim 9, wherein the step of determining whether to output the warning message comprises:
when remaining in the third state and an actual average distance between every two peak occurring time points is smaller than a determined distance, outputting a warning message to warn that the dropping frequency is too high.

11. The intravenous drip monitoring method of claim 9, wherein the step of determining whether to output the warning message comprises:
when remaining in the third state and an actual average distance between every two peak occurring time points is greater than a determined distance, outputting a warning message to warn that the dropping frequency is too low.

12. The intravenous drip monitoring method of claim 9, wherein the step of determining whether to output the warning message comprises:
when entering to the fourth state from the first state, outputting a warning message to warn that the intravenous drip stops to drip.

13. The intravenous drip monitoring method of claim 9, wherein the step of determining whether to output the warning message comprises:
when entering to the fourth state from the first state and the liquid horizontal height of the flow regulating device is lower than a determined height, outputting a warning message to warn that the intravenous drip is empty.

14. The intravenous drip monitoring method of claim 9, wherein the step of determining whether to output the warning message comprises:
when entering to the fourth state from the first state and the liquid horizontal height of the flow regulating device does not have any variation, outputting a warning message to warn that a tube of the intravenous drip is jammed.

15. The intravenous drip monitoring method of claim 4, wherein the step of utilizing the brightness variations generated by the motion drips in the flow regulating device to detect the dropping frequency of the intravenous drip in a first detecting area of the flow regulating device further comprises:
Observing the numbers generated by the motion drips in the flow regulating device passing the detecting sub-area, and setting the threshold accordingly.

16. The intravenous drip monitoring method of claim 1, wherein the step of utilizing the brightness variations generated by the motion drips in the flow regulating device to detect the liquid horizontal height of the flow regulating device in a second detecting area of the flow regulating device comprises:
defining a plurality of detecting sub-areas in the second detecting area from top to bottom;
observing numbers of motion blocks generated by the motion drips of the flow regulating device passing through the plurality of detecting sub-areas during a specific period;
when observing the numbers of motion blocks in a detecting sub-area of the plurality of detecting sub-areas exceeding a threshold, updating a number of peak accumulating times corresponding to the detecting sub-area; and
determining the liquid horizontal height of the flow regulating device in accordance with a plurality of numbers of peak accumulating times corresponding to at least the plurality of detecting sub-areas.

17. The intravenous drip monitoring method of claim 16, wherein the step of determining the liquid horizontal height of the flow regulating device comprises:
performing a weighted averages process to determine the liquid horizontal height of the flow regulating device in accordance with the plurality of numbers of peak accumulating times and heights of the plurality of detecting sub-areas.

18. The intravenous drip monitoring method of claim 16, wherein the step of determining the liquid horizontal height of the flow regulating device comprises:
determining the liquid horizontal height of the flow regulating device in accordance with a detecting sub-area having a largest peak accumulating time of the plurality of detecting sub-areas.

19. An intravenous drip monitoring system, comprising:
an image obtaining device, positioned in a side of a flow regulating device of an intravenous drip, utilized for obtaining a plurality of frames in the flow regulating device of the intravenous drip;
a color plate, positioned in a side of a flow regulating device of an intravenous drip, utilized for providing a background color as a background of a flow regulating device of an intravenous drip; and
an image processing device, coupled to the image obtaining device, utilized for:
obtaining a plurality of frames in the flow regulating device of the intravenous drip in accordance of a frame rate;
performing an image processing operation on the plurality of frames to obtain brightness variations generated by motion drips in the flow regulating device;
utilizing the brightness variations generated by the motion drips in the flow regulating device to detect a dropping frequency of the intravenous drip in a first detecting area of the flow regulating device; and
utilizing the brightness variations generated by the motion drips in the flow regulating device to detect a liquid horizontal height of the flow regulating device in a second detecting area of the flow regulating device.

20. The intravenous drip monitoring system of claim 19, wherein the image processing device is further utilized for:
utilizing the brightness variations generated by the motion drips in the flow regulating device to define the first detecting area and the second detecting area.

21. The intravenous drip monitoring system of claim 20, wherein the function of the image processing device for utilizing the brightness variations generated by the motion drips in the flow regulating device to define the first detecting area and the second detecting area comprises:
utilizing the brightness variations generated by the motion drips in the flow regulating device to find a drip dropping starting point in an upper area of the flow regulating device;
defining an area around the drip dropping starting point as the first detecting area;
utilizing the brightness variations generated by the motion drips in the flow regulating device to find a liquid surface in a lower area of the flow regulating device; and
defining an area around the water surface as the second detecting area.

22. The intravenous drip monitoring system of claim 19, wherein the function of the image processing device for utilizing the brightness variations generated by the motion drips in the flow regulating device to detect the dropping frequency of the intravenous drip in a first detecting area of the flow regulating device comprises:
defining at least a detecting sub-area in the first detecting area;
detecting numbers of motion blocks in the detecting sub-area;
when observing the numbers of motion blocks respectively exceeding a threshold at a plurality of time points, labeling the plurality of time points as a plurality of peak occurring time points;
calculating an average distance between the plurality of peak occurring time points; and
detecting the dropping frequency of the intravenous drip in accordance with the average distance and following peak occurring time points.

23. The intravenous drip monitoring system of claim 22, wherein the function of the image processing device for detecting the dropping frequency of the intravenous drip in accordance with the average distance and the following peak occurring time points comprises:
setting a first observing window at each time point spaced at an interval of the average distance;
in a first state, determining whether a peak occurring time point appears in a current first observing window, wherein:
when the peak occurring time point does not appear in the current first observing window, keeping in the first state and adjusting the frame rate, and calculating the average distance again; and
when the peak occurring time point appears in the current first observing window, entering a second state;
in the second state, determining whether a peak occurring time point appears in a current first observing window and determining whether a first number of accumulating times of the peak occurring time points greater than a first predetermined number, wherein:
when the peak occurring time point does not appear in the current first observing window, go back to the first state and calculating the average distance again;
when the peak occurring time point appears in the current first observing window, and the first number of accumulating times of the peak occurring time points is smaller than the first predetermined number, keeping in the second state; and
when the peak occurring time point appears in the current first observing window, and the first number of accumulating times of the peak occurring time points is not smaller than the first predetermined number, entering a third state;
in the third state, calculating an actual distance per two peak occurring time points to calculating an actual average distance per two peak occurring time points, setting a second observing window at each time point spaced at an interval of the actual average distance, and determining whether a peak occurring time point appears in a current second observing window, wherein:
when the peak occurring time point does not appear in the current second observing window, entering a fourth state; and
when the peak occurring time point appears in the current second observing window, keeping in the third state;
in the fourth state, determining whether a peak occurring time point appears in a current second observing window and determining whether a second number of accumulating times of the peak occurring time points greater than a second predetermined number, wherein:
when the peak occurring time point does not appear in the current second observing window, go back to the third state;
when the peak occurring time point does not appear in the current second observing window, and the second number of accumulating times of the peak occurring time points is greater than the second predetermined number, entering the first state; and
when the peak occurring time point does not appear in the current second observing window, and the second number of accumulating times of the peak occurring time points is not greater than the second predetermined number, keeping in the fourth state.

24. The intravenous drip monitoring system of claim 23, wherein the function of the image processing device for utilizing the brightness variations generated by the motion drips in the flow regulating device to detect the liquid horizontal height of the flow regulating device in a second detecting area of the flow regulating device comprises:
defining a plurality of detecting sub-areas in the second detecting area from top to bottom;
observing numbers of motion blocks generated by the motion drips of the flow regulating device passing through the plurality of detecting sub-areas during a specific period;
when observing the numbers of motion blocks in a detecting sub-area of the plurality of detecting sub-areas exceeding a threshold, updating a number of peak accumulating times corresponding to the detecting sub-area; and
determining the liquid horizontal height of the flow regulating device in accordance with a plurality of numbers of peak accumulating times corresponding to at least the plurality of detecting sub-areas.

25. The intravenous drip monitoring system of claim 24, wherein the function of the image processing device for determining the liquid horizontal height of the flow regulating device comprises:
performing a weighted averages process to determine the liquid horizontal height of the flow regulating device in accordance with the plurality of numbers of peak accumulating times and heights of the plurality of detecting sub-areas.

26. The intravenous drip monitoring system of claim 24, wherein the function of the image processing device for determining the liquid horizontal height of the flow regulating device comprises:
determining the liquid horizontal height of the flow regulating device in accordance with a detecting sub-area having a largest peak accumulating time of the plurality of detecting sub-areas.

27. The intravenous drip monitoring system of claim 24, wherein the image processing device is further utilized for:
determining whether to output a warning message in accordance with the dropping frequency and the liquid horizontal height.

28. The intravenous drip monitoring system of claim 27, wherein the function of the image processing device for determining whether to output the warning message comprises:
when remaining in the third state and an actual average distance between every two peak occurring time points is smaller than a determined distance, outputting a warning message to warn that the dropping frequency is too high.

29. The intravenous drip monitoring system of claim 27, wherein the function of the image processing device for determining whether to output the warning message comprises:
when remaining in the third state and an actual average distance between every two peak occurring time points is greater than a determined distance, outputting a warning message to warn that the dropping frequency is too low.

30. The intravenous drip monitoring system of claim 27, wherein the function of the image processing device for determining whether to output the warning message comprises:
when entering to the fourth state from the first state, outputting a warning message to warn that the intravenous drip stops to drip.

31. The intravenous drip monitoring system of claim 27, wherein the function of the image processing device for determining whether to output the warning message comprises:
when entering to the fourth state from the first state and the liquid horizontal height of the flow regulating device is lower than a determined height, outputting a warning message to warn that the intravenous drip is empty.

32. The intravenous drip monitoring system of claim 27, wherein the function of the image processing device for determining whether to output the warning message comprises:
when entering to the fourth state from the first state and the liquid horizontal height of the flow regulating device does not have any variation, outputting a warning message to warn that a tube of the intravenous drip is jammed.

33. The intravenous drip monitoring system of claim 23, wherein the function of the image processing device for utilizing the brightness variations generated by the motion drips in the flow regulating device to detect the dropping frequency of the intravenous drip in a first detecting area of the flow regulating device further comprises:
Observing the numbers generated by the motion drips in the flow regulating device passing the detecting sub-area, and setting the threshold accordingly.

34. The intravenous drip monitoring system of claim 19, wherein the function of the image processing device for utilizing the brightness variations generated by the motion drips in the flow regulating device to detect the liquid horizontal height of the flow regulating device in a second detecting area of the flow regulating device comprises:
defining a plurality of detecting sub-areas in the second detecting area from top to bottom;
observing numbers of motion blocks generated by the motion drips of the flow regulating device passing through the plurality of detecting sub-areas during a specific period;
when observing the numbers of motion blocks in a detecting sub-area of the plurality of detecting sub-areas exceeding a threshold, updating a number of peak accumulating times corresponding to the detecting sub-area; and
determining the liquid horizontal height of the flow regulating device in accordance with a plurality of numbers of peak accumulating times corresponding to at least the plurality of detecting sub-areas.

35. The intravenous drip monitoring system of claim 34, wherein the function of the image processing device for determining the liquid horizontal height of the flow regulating device comprises:
performing a weighted averages process to determine the liquid horizontal height of the flow regulating device in accordance with the plurality of numbers of peak accumulating times and heights of the plurality of detecting sub-areas.

36. The intravenous drip monitoring system of claim 34, wherein the function of the image processing device for determining the liquid horizontal height of the flow regulating device comprises:
determining the liquid horizontal height of the flow regulating device in accordance with a detecting sub-area having a largest peak accumulating time of the plurality of detecting sub-areas.

37. The intravenous drip monitoring system of claim 19, wherein the image processing device is a digital camera or a web cam.
